(19) United States
(12) Patent Application Publication
(10) Pub. No.: US 2026/0179742 A1
(43) Pub. Date: Jun. 25, 2026

(54) SYSTEM AND METHOD FOR MANAGING TREATMENT PLANS AND CLINICAL DOCUMENTATION

(71) Applicants: Roderick L Brown, PE, Southfield, MI (US); Dwayne E Goldsmith, PE, Northville, MI (US); Vassal N Johnson, Southfield, MI (US)

(72) Inventors: Roderick L Brown, PE, Southfield, MI (US); Dwayne E Goldsmith, PE, Northville, MI (US); Vassal N Johnson, Southfield, MI (US)

(73) Assignee: ASENDIO, Inc, Ann Arbor, MI (US)

(21) Appl. No.: 19/434,278

(22) Filed: Dec. 29, 2025

Related U.S. Application Data
(60) Provisional application No. 63/738,680, filed on Dec. 24, 2024.

(51) Int. Cl. G16H 10/60; G16H 15/00

(52) U.S. Cl. CPC G16H 10/60; G16H 15/00

(57) ABSTRACT

The present invention discloses a system and method for managing treatment plans and clinical documentation for substance use disorder (SUD) treatment. The system includes user devices associated with clients, clinicians, organization representatives, and supervisors. The system connects to a database storing client data, clinician data, assessment scores, and clinical documentation such as SOAP notes and progress notes. A computing device with artificial intelligence (AI) modules executes various program modules. A data collection module guides clinicians through predefined questions to gather information for generating SOAP and clinical progress notes. A secondary data collection module collects clarification from the counselor without requiring client input. A review module allows counselors to review, edit, approve, and submit documentation. A treatment plan generation module creates or updates plans based on collected data and prior scores. The system improves documentation efficiency, quality, and compliance, while supporting accurate and personalized treatment planning.

### CROSS REFERENCE TO RELATED APPLICATION

This application claims the priority benefit of the U.S. Patent Application No. 63/738,680 titled “SYSTEM AND METHOD FOR TREATING SUBSTANCE USE DISORDER”, filed on Dec. 24, 2024, the contents of which are hereby incorporated by reference in their entirety.

### TECHNICAL FIELD

The present invention generally relates to management of treatment plans and clinical documentation. More particularly, the invention is related to a system and method for managing treatment plans and clinical documentation for substance use disorder (SUD) treatment.

### BACKGROUND

Substance Use Disorder (SUD), a complex medical condition in which an individual has a diminished ability to control the use of one or more legal or illegal substances. Further, the substances could be alcohol, drugs, or prescription medications. The SUD further involves a range of behavioral, cognitive, and physiological issues including cravings, inability to control substance use, and withdrawal symptoms. Further, repeated usage of substances could affect the brain function system.

SUD could vary in severity from mild to severe with mental health disorders, for example, anxiety or depression. The disorder could impact people and lead to significant personal, social, and economic consequences. Further, the SUD needs to be monitored to provide the required support to manage the potential risk of SUD individuals. Further, a few patents reference related to treating substance use disorder (SUD) are discussed as follows.

US20210335489 of Dennis Wall entitled “Enhancing diagnosis of disorder through artificial intelligence and mobile health technologies without compromising accuracy” discloses a computer system for generating a diagnostic tool by applying artificial intelligence to an instrument designed for the diagnosis of disorders, such as autism. The instrument is a caregiver-directed set of questions designed for an autism classification tool or an observation of the subject in a video, video conference, or in person and associated set of questions about behavior that are designed for use in a separate autism classification tool. The system further comprises one or more processors and memory configured to store computer programs with instructions for generating a statistically accurate set of diagnostic items derived from the instrument. The diagnostic items are validated through a first test employing artificial intelligence techniques and a second test conducted against an independent source.

US20190019581 of Brent Vaughan et. al., entitled “Platform and system for digital personalized medicine” discloses a method and apparatus to provide digital diagnostics and digital therapeutics to patients. The system assesses or diagnoses patient symptoms and incorporates feedback from the patient's response to treatment to update personalized therapeutic interventions. The methods and apparatus are designed to diagnose and treat cognitive function with few questions, and limited time. Further, the method enables to determine a plurality of behavioral, neurological or mental health disorders by maintaining clinically acceptable sensitivity and specificity.

However, the existing system fails to provide quantitative data to monitor the health of individuals with SUD across different counselors and organizations. Further, the system lacks the ability to evaluate the overall health of SUD individuals across various organizations to provide a treatment plan. The system lacks to provide comprehensive data of SDU individual which might complicate the understanding of a client's progress.

Therefore, there is a need for a system and method for managing treatment plans and clinical documentation for substance use disorder (SUD) treatment. Further, the system needs to track the overall health of the SUD individual to optimize treatment plans. Further, the system needs to provide comprehensive data to help the clinician and client to understand the client's progress and provide a proper treatment plan.

### SUMMARY

The present invention discloses a system and method for managing treatment plans and clinical documentation for substance use disorder (SUD) treatment. The system comprises one or more user devices including at least one first user device associated with a client, at least one second user device associated with a clinician, at least one third user device associated with a representative of an organization and at least one fourth user device associated with a supervisor. The system further comprises at least one database storing a client data, a clinician data, a score data of each client assessment, and data related to clinical documentation, including SOAP notes and clinical progress notes. The system further comprises at least one computing device in communication with at least one database and the user devices via a network.

The computing device comprises one or more artificial intelligence (AI) modules. The computing device comprises at least one memory configured to store a set of program modules and at least one processor configured to execute one or more program modules. The modules comprise a data collection module configured to present a predefined first set of questions at the second user device and collect information for generating SOAP notes. The data collection module is further configured to present a predefined second set of questions and collect information for generating clinical progress notes. The data collection module is configured to generate the clinical progress note and SOAP note using a predefined style guide, grammar enhancement tools, and one or more evidence-based documentation inputs.

The modules further comprise a secondary data collection module configured to present a predefined third set of questions. The third set of questions are configured to collect information to elicit clarification from the counselor without requiring a response from the client.

The modules further comprise a review module configured to present generated SOAP notes and clinical progress notes and configured to enable the counselor to customize the notes through one or more options, including review, and edit, and enable the counselor to approve, and submit the SOAP notes and clinical progress notes.

The modules further comprise a documentation module configured to improve grammar, structure, and style, without altering a content of the information received from the counselor.

The modules further comprise a treatment plan generation module configured to generate a treatment plan based on information received in response to the set of questions and any existing treatment plan or associated score data. The treatment plan generation module is further configured to utilize clinical progress notes and score data to provide one or more treatment plan modifications to the counselor for incorporation into the treatment plan. The treatment plan is for a predefined duration based on the information of the client including, score data and clinical progress notes.

The modules further comprise a coaching document generation module configured, upon request, to provide a supervisor with documentation feedback and improvement suggestions related to clinical note quality, formatting, and compliance. The feedback is being generated to enable the supervisor to guide the counselor.

The modules further comprise a feedback module configured to analyze clinical progress notes and provide feedback to the counselor for improving the quality, structure, and compliance of the clinical progress notes.

The modules further comprise a query module configured to provide a questionnaire comprising a plurality of categories. Each category comprises a set of questions to assess progress of the client. The query module is configured to enable the clinician to input a score for each question of each category based on analyzing response of client to each question.

The modules further comprise a scoring module configured to apply predefined scoring guidelines for the input score and generate the score data. The modules further comprise a personal recovery plan module configured to generate a client-specific strategic recovery document. The recovery document is a persistent, client-owned record transferable across counselling sessions and service providers. The personal recovery plan module is further configured to enable client participation in treatment planning using the recovery document.

The modules further comprise an assessment analysis module configured to analyze score data associated with the client and generate one or more recommendations for influencing a plurality of variables of the questionnaire.

The modules further comprise a personal recovery assistant module configured to monitor score data associated with the client to detect indicators of non-compliance with recovery protocols, and to provide the client with personalized feedback and suggestions for improving adherence to the treatment plan.

In one embodiment, a method for managing treatment plans and clinical documentation is disclosed. The method is executed by a system comprising one or more user devices, a database, and at least one computing device in communication with the database and the user devices via a network. The user devices include at least one first user device associated with a client, at least one second user device associated with a clinician, at least one third user device associated with a representative of an organization, and at least one fourth user device associated with a supervisor. The database comprises client data, clinician data, score data of each client assessment, and data related to clinical documentation, including SOAP notes and clinical progress notes.

The computing device comprises one or more artificial intelligence (AI) modules. The computing device comprises at least one memory configured to store a set of program modules and at least one processor configured to execute one or more program modules. The program modules include a data collection module, a secondary data collection module, a review module, a documentation module, a treatment plan generation module, a coaching document generation module, a feedback module, a query module, a scoring module, a personal recovery plan module, an assessment analysis module, and a personal recovery assistant module.

At one step, the data collection module, at the computing device, is configured to present a predefined first set of questions at the second user device and collect information for generating SOAP notes. The data collection module is further configured to present a predefined second set of questions and collect information for generating clinical progress notes. The data collection module generates the SOAP notes and clinical progress notes using a predefined style guide, grammar enhancement tools, and one or more evidence-based documentation inputs.

At yet another step, the secondary data collection module, at the computing device, is configured to present a predefined third set of questions to elicit clarification from the counselor without requiring a response from the client.

At yet another step, the review module, at the computing device, is configured to present the generated SOAP notes and clinical progress notes to the counselor, and enable customization through one or more options, including review and edit, and to approve and submit the SOAP notes and clinical progress notes.

At yet another step, the documentation module, at the computing device, is configured to improve grammar, structure, and style of the SOAP notes and clinical progress notes without altering the content of the information received from the counselor.

At yet another step, the treatment plan generation module, at the computing device, is configured to generate a treatment plan based on information received in response to the sets of questions and any existing treatment plan or associated score data. The treatment plan generation module further utilizes clinical progress notes and score data to provide one or more treatment plan modifications to the counselor for incorporation into the treatment plan. The treatment plan is for a predefined duration based on the information of the client including score data and clinical progress notes.

At yet another step, the coaching document generation module, at the computing device, is configured to generate documentation feedback and improvement suggestions related to clinical note quality, formatting, and compliance, and enable the supervisor to guide the counselor.

At yet another step, the feedback module, at the computing device, is configured to analyze the clinical progress notes and provide feedback to the counselor for improving the quality, structure, and compliance of the notes. At yet another step, the query module, at the computing device, is configured to provide a questionnaire comprising a plurality of categories. Each category comprises a set of questions to assess progress of the client. The query module is further configured to enable the clinician to input a score for each question of each category based on analysing the response of the client to each question. At yet another step, the scoring module is configured to apply predefined scoring guidelines for the input score to generate the score data.

At yet another step, the personal recovery plan module, at the computing device, is configured to generate a client-specific strategic recovery document. The recovery document is a persistent, client-owned record transferable across counselling sessions and service providers, and further configured to enable client participation in treatment planning using the recovery document. At yet another step, the assessment analysis module is configured to analyze score data associated with the client and generate one or more recommendations for influencing a plurality of variables of the questionnaire. At yet another step, the personal recovery assistant module is configured to monitor the score data associated with the client to detect indicators of non-compliance with recovery protocols and to provide the client with personalized feedback and suggestions for improving adherence to the treatment plan.

Other objects, features and advantages of the present innovation will become apparent from the following detailed description. It should be understood, however, that the detailed description and the specific examples, while indicating specific embodiments of the innovation, are given by way of illustration only, since various changes and modifications within the spirit and scope of the innovation will become apparent to those skilled in the art from this detailed description.

### BRIEF DESCRIPTION OF THE DRAWINGS

The foregoing summary, as well as the following detailed description of the innovation, is better understood when read in conjunction with the appended drawings. For the purpose of illustrating the innovation, exemplary constructions of the innovation are shown in the drawings. However, the innovation is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates an environment of a system for managing treatment plans and clinical documentation for substance use disorder (SUD) treatment, according to an embodiment of the present invention.

FIG. 2 exemplarily illustrates a block diagram of the computing device, according to an embodiment of the present invention.

FIG. 3 exemplarily illustrates a flowchart of a method for managing treatment plans and clinical documentation for substance use disorder (SUD) treatment, according to an embodiment of the present invention.

### DETAILED DESCRIPTION OF EXAMPLE EMBODIMENTS

A description of embodiments of the present innovation will now be given with reference to the Figures. It is expected that the present innovation may be embodied in other specific forms without departing from its spirit or essential characteristics. The described embodiments are to be considered in all respects only as illustrative and not restrictive.

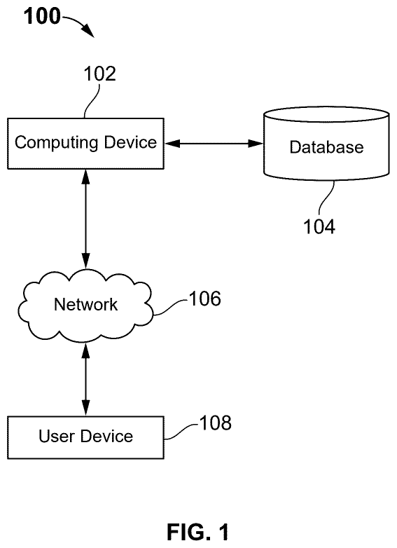

FIG. 1 exemplarily illustrates an environment of a system 100 for managing treatment plans and clinical documentation for substance use disorder (SUD) treatment, according to an embodiment of the present invention. The system 100 comprises at least one computing device 102, at least one database 104 in communication with the computing device 102. The system 100 further comprises at least one user device 108 associated with a user. The user device 108 and the database 104 are in communication with the computing device 102 via a network 106.

The user device 108 is configured to provide an interface to access the services provided by the computing device 102. The interface, for example, an application that allows the user device 108 to wirelessly connect and access the computing device 102 via the network 106. Further, the interface could also be a cloud-based platform or a web-based application. The cloud-based platform includes, but not limited to, amazon web services (AWS) and Microsoft azure. The AWS and azure environments are configured with enhanced security protocols, including data encryption in transit and at rest, identity and access management, and regular security audits. Further, the protocols ensure adherence to regulatory standards set by federal and state laws for patient data protection. The user device 108 includes, but not limited to, a desktop computer, a laptop computer, a mobile phone, a personal digital assistant, and the like. The user could be a clinician or a client related to substance use disorder (SUD). In one example, the system 100 is used in an android, an iOS, and a window. The user devices 108 include at least one first user device associated with a client, at least one second user device associated with a clinician, at least one third user device associated with a representative of an organization, and at least one fourth user device associated with a supervisor.

The user device 108 is configured to provide one or more client data. The client is an individual related to substance use disorder (SUD). The client data is provided based on three categories. The three categories comprise a revive, a restore, a renew. Each category comprises one or more scoring guide inputs. Further, each scoring guide input is assigned a score value. The score value ranges from 1-10. The scoring system based on the revive, restore, and renew categories helps to tailor personalized treatment plans for each client.

The revive category provides one or more information related to the housing stability, the dosing consistency, and the illicit substance. The housing stability is evaluated based on the information related to the housing stability status of the client. The dosing consistency is evaluated based on the information related to the number of doses missed in the past 30 days by the client. Further, the illicit substance is evaluated based on the information related to the amount of illicit substance usage by the client.

The restore category provides one or more information related to the counseling consistency, the physical and mental health, and the legal issues impact. The counseling consistency is evaluated based on the information related to the number of sessions missed in the past three months by the client. The physical and mental health is evaluated based on the information related to the physical and mental health of the client. Further, the legal issues impact is evaluated based on the information related to the legal issues involved by the client.

The renew category provides one or more information related to the renew gap, the interpersonal relationship, and the medication management. The renew gap is evaluated based on the information related to the number of questions the client answered with “No” in relation to client information. The interpersonal relationship is evaluated based on the information related to the relationship between the client and other people from family or friends. Further, the medication management is evaluated based on the information related to the duration taken continuous treatment and free from the illicit drug in the client. Further, a score is assigned by the computing device 102 by analyzing the information provided by the user via user device 108 for the revive category, the restore category, the renew category.

The network 106 generally represents one or more interconnected networks, over which the user device 108, the computing device 102 could communicate with each other. The network 106 may include packet-based wide area networks (such as the Internet), local area networks (LAN), private networks, wireless networks, satellite networks, cellular networks, paging networks, and the like. A person skilled in the art will recognize that the network 106 may also be a combination of more than one type of network. For example, the network 106 may be a combination of a LAN and the Internet. In addition, the network 106 may be implemented as a wired network or a wireless network or a combination thereof.

The system 100 further comprises at least one database 104 in communication with the computing device 102. In an example, the database 104 resides in the computing device 102. In another example, the database 104 resides separately from the computing device 102. Regardless of the location, the database 104 comprises a memory to store and organize data for use by the computing device 102. The database 104 comprises information related to the client with a substance use disorder (SUD) for use by the computing device 102. The database 104 stores a client data, a clinician data, a score data of each client assessment, and data related to clinical documentation, including SOAP notes and clinical progress notes.

The computing device 102 and the database 104 are in communication with the computing device 102 via the network 106. In one embodiment, the computing device 102 is at least one of a server, a general-purpose computer, a special-purpose computer, a workstation, a desktop, a laptop, a tablet, a mobile phone, a mainframe, a supercomputer and a server farm. Although the computing device 102 is illustrated as a single device, the functions performed by the computing device 102 could be performed using any suitable number of computing devices 102.

The computing device 102 comprises an artificial intelligence (AI) module. The artificial intelligence (AI) module helps in improving the capabilities to perform tasks, making decisions, and solving problems. Further, the AI module helps to support substance use disorder (SUD) treatment. The AI module further enhances personalization of treatment plan by leveraging detailed patient inputs and scoring input.

The AI tool or modules comprises an open-source large language models (LLMs) fine-tuned with domain-specific knowledge related to substance use disorder (SUD) treatment. Further, the AI tools comprise one or more training data sources. The training data source includes guidelines and one or more policies from federal agencies, for example, the substance abuse and mental health services administration (SAMHSA) and drug enforcement administration (DEA). Further, the training data source also comprises industry-recognized evidence-based practices and state-level regulations, for example.

The computing device 102 is configured to receive one or more input data from the user via the user device 108. The input data is a self-assessment of the client provided by the user. The computing device 102 comprises the AI module configured to analyze the input data for any grammatical errors, phrasing, or structure and style to enhance the input data quality. The AI module further applies stylistic improvement and standardized formatting to the input data and ensures the text is clear without changing the content.

The computing device 102 is configured to provide one or more queries after each counseling session is completed. Further, the computing device 102 is configured to request the responses for the queries from a counselor. Further, the counselor needs to provide clarification that require no response from the client. Further, the queries are generally answerable if the counselor has conducted the session in a professional manner. The computing device 102 is configured to assist the counselor in generating a subjective, objective, assessment, and plan (SOAP) note using the AI tool. Further, the computing device 102 is configured to provide a ten-question guide to the counselor and collect the required information to complete the detailed SOAP note. The computing device 102 is further configured to standardize the SOAP notes using a style guide, grammatical tools, and other evidence-based input. Further, the computing device 102 comprises an automated compliance tool, for example, SOAP note generation, to facilitate the clinical workflows.

The computing device 102 is further configured to enable the counselor to review a standardized SOAP note. The review process helps the counselor to verify that the session information aligns with the client's current status. Further, the computing device 102 is configured to enable the counselor to edit the SOAP notes. The editing process ensures the session information aligns with the client's current status. Further, the computing device 102 is configured to enable the counselor to approve and submit the SOAP notes. The approval process ensures that the SOAP notes are stored for further process.

The computing device 102 is configured to assist the counselor in generating a clinical progress note using the AI tool. Further, the computing device 102 tool is configured to provide a twenty-question guide to the counselor and collect the comprehensive data required for the clinical progress note. The computing device 102 is further configured to standardize comprehensive data required for the clinical progress note using a style guide, grammatical tools, and other evidence-based guidelines. The computing device 102 is further configured to enable the counselor to review, edit, approve, and submit the finalized clinical progress note.

The computing device 102 is further configured to generate a consolidated summary from the SOAP note and the clinical progress note. The consolidated summary provides a review over 90 days. The 90-day review enables the counselor or clinician to assess the client's progress in detail. Further, the 90-day review helps to provide a treatment plan to the client. The computing device 102 is further configured to receive an update from the user for the treatment plan. The updates could be significant progress or challenges faced by the client. Further, the treatment plan is adjusted to address the client's current situation.

The computing device 102 is configured to create a reference treatment plan using the AI tools. Further, the reference treatment plan is compared with the existing treatment plan. Further, the treatment plan is integrated with the input comprising score data and the current information of the client from consolidated summary. Further, the consolidated summary is used to provide a treatment plan for 3, 6, 16, or 24 months in duration.

The computing device 102 is further configured to provide a revised treatment plan based on the updated clinical progress notes and the updated scores assigned to the client data. The clinical progress notes and score data are used for adjusting the treatment plan for the client. Further, the score data is reviewed using the AI tool to provide the counselor with strategies to adjust the revised treatment plan.

The computing device 102 is further configured to provide a set of recommendations for the counselor. Further, the counselor analyses the recommendation and the revised treatment plan to ensure that the revised treatment plan is aligned with the client's current needs.

The computing device 102 is further configured to provide a clinical coaching document using the AI tool. The clinical coaching document is provided upon request from the counselor. The clinical coaching document comprises a note and improvised suggestions. Further, the clinical coaching document is designed to enhance the counselor's performance. Further, the computing device 102 is configured to receive updated counselor's note from the counselor.

The computing device 102 is further configured to review the counselor's notes to provide feedback in real-time. The real-time feedback provides the counselors with suggestions to enhance the quality of the counselor notes.

The computing device 102 is further configured to provide a private personal treatment plan or recovery treatment plan for the client. The private personal treatment plan provides an outline of a total recovery strategy. The outline of the total recovery strategy includes, but not limited to progress metrics, and specific recovery strategies. The recovery strategy engages the clients to participate actively in the treatment planning process. Further, the private personal treatment plan could be shared across different counselors and agencies to ensure consistent support and continuity of care for long-term recovery.

The computing device 102 is further configured to assist the AI tool in monitoring the client non-compliance using score data. The non-compliance includes the client who missed counseling sessions or delays in progress. Further, the computing device 102 is configured to provide the clients with feedback and suggestions to improve adherence to recovery approaches.

The computing device 102 is further configured to maintain and assess the quality of documentation using the AI tool. Further, the AI tool detects and prevents the duplication of the documentation across different clients, counselors, or sessions. Further, the AI tool ensures accuracy and protects the integrity of the documentation.

The system 100 is further integrated with existing electronic health records (EHR) systems via a secure application programming interface (APIs), for example, Methasoft. Further, the integration ensures the real-time synchronization of client data across multiple platforms.

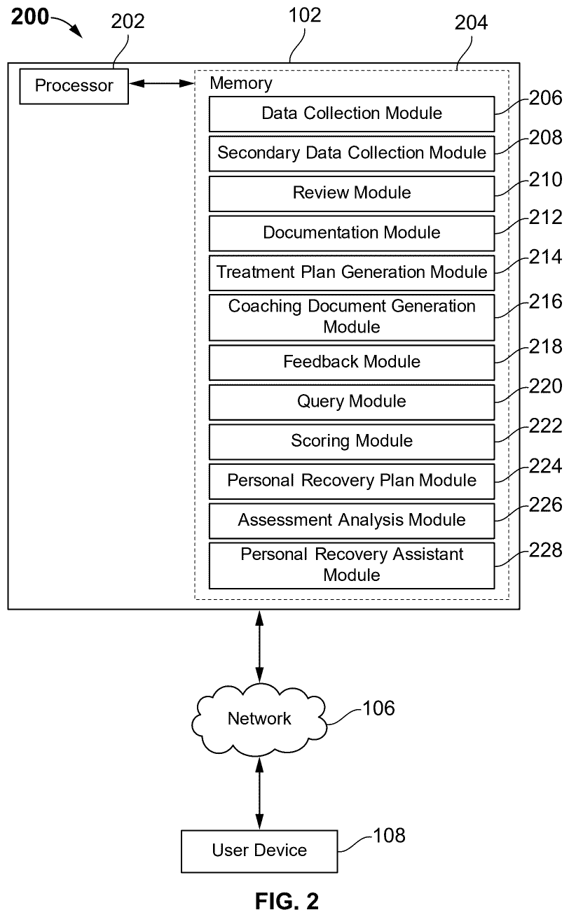

FIG. 2 exemplarily illustrates a block diagram 200 of the computing device 102 of the system 100, according to an embodiment of the present invention. The computing device 102 comprises one or more processors 202 and a memory 204 storing set of program modules. The processor 202 is configured to execute the program modules. The processor 202 is configured to execute one or more program modules.

The modules comprise a data collection module 206 configured to present a predefined first set of questions at the second user device and collect information for generating SOAP notes. The data collection module 206 is further configured to present a predefined second set of questions and collect information for generating clinical progress notes. The data collection module 206 is configured to generate the clinical progress note and SOAP note using a predefined style guide, grammar enhancement tools, and one or more evidence-based documentation inputs.

The modules further comprise a secondary data collection module 208 configured to present a predefined third set of questions. The third set of questions are configured for collecting information to elicit clarification from the counselor without requiring a response from the client. The questions are answerable if the counselor has conducted the session in a professional manner.

The modules further comprise a review module 210 configured to present generated SOAP notes and clinical progress notes and configured to enable the counselor to customize the notes through one or more options including review, and edit. The review module 210 enables the counselor to approve, and submit the SOAP notes and clinical progress notes. The modules further comprise a documentation module 212 configured to improve grammar, structure, and style, without altering a content of the information received from the counselor.

The modules further comprise a treatment plan generation module 214 configured to generate a treatment plan based on information received in response to the set of questions and any existing treatment plan or associated score data. The treatment plan generation module 214 is further configured to utilize clinical progress notes and score data to provide one or more treatment plan modifications to the counselor for incorporation into the treatment plan. The treatment plan is for a predefined duration based on the information of the client including score data and clinical progress notes.

The modules further comprise a coaching document generation module 216 configured, upon request, to provide a supervisor with documentation feedback and improvement suggestions related to clinical note quality, formatting, and compliance. The feedback is being generated to enable the supervisor to guide the counselor.

The modules further comprise a feedback module 218 configured to analyze clinical progress notes and provide feedback to the counselor for improving the quality, structure, and compliance of the clinical progress notes. The modules further comprise a query module 220 configured to provide a questionnaire comprising a plurality of categories. Each category comprises a set of questions to assess progress of the client. The query module 220 is configured to enable the clinician to input a score for each question of each category based on analyzing response of client to each question. The modules further comprise a scoring module 222 configured to apply predefined scoring guidelines for the input score and generate the score data.

The modules further comprise a personal recovery plan module 224 configured to generate a client-specific strategic recovery document. The recovery document is a persistent, client-owned record transferable across counseling sessions and service providers. The personal recovery plan module 224 is further configured to enable client participation in treatment planning using the recovery document.

The modules further comprise an assessment analysis module 226 configured to analyze score data associated with the client and generate one or more recommendations for influencing a plurality of variables of the questionnaire.

The modules further comprise a personal recovery assistant module 228 configured to monitor score data associated with the client to detect indicators of non-compliance with recovery protocols, and to provide the client with personalized feedback and suggestions for improving adherence to the treatment plan. The computing device 102 comprises an artificial intelligence (AI)-driven quality assurance mechanism configured to detect and prevent duplication of documentation across multiple clients, counselors, or sessions. The computing device 102 is further configured to identify and present both similarities and differences between two distinct clinical notes.

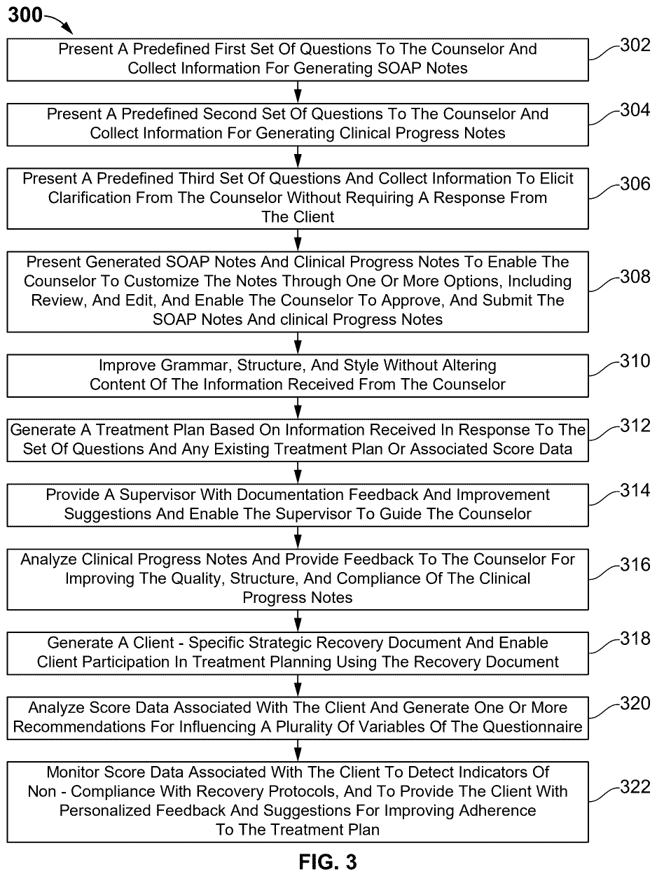

FIG. 3 exemplarily illustrates a flowchart of a method 300 for managing treatment plans and clinical documentation, according to an embodiment of the present invention. The method 300 is executed by a system comprising one or more user devices 108, a database 104, and at least one computing device 102 in communication with the database 104 and the user devices 108 via the network 106. The user devices 108 include at least one first user device associated with a client, at least one second user device associated with a clinician, at least one third user device associated with a representative of an organization, and at least one fourth user device associated with a supervisor. The database 104 comprises client data, clinician data, score data of each client assessment, and data related to clinical documentation including SOAP notes and clinical progress notes.

The computing device 102 comprises one or more artificial intelligence (AI) modules. The computing device 102 comprises at least one memory 204 configured to store a set of program modules and at least one processor 202 configured to execute one or more program modules. The program modules include a data collection module 206, a secondary data collection module 208, a review module 210, a documentation module 212, a treatment plan generation module 214, a coaching document generation module 216, a feedback module 218, a query module 220, a scoring module 222, a personal recovery plan module 224, an assessment analysis module 226, and a personal recovery assistant module 228.

At step 302, the data collection module 206, at the computing device 102, is configured to present a predefined first set of questions at the second user device and collect information for generating SOAP notes. At step 304, the data collection module 206 is further configured to present a predefined second set of questions and collect information for generating clinical progress notes. The data collection module 206 generates the SOAP notes and clinical progress notes using a predefined style guide, grammar enhancement tools, and one or more evidence-based documentation inputs.

At step 306, the secondary data collection module 208, at the computing device 102, is configured to present a predefined third set of questions to elicit clarification from the counselor without requiring a response from the client.

At step 308, the review module 210, at the computing device 102, is configured to present the generated SOAP notes and clinical progress notes to the counselor, and enable customization through one or more options including review and edit, and to approve and submit the SOAP notes and clinical progress notes.

At step 310, the documentation module 212, at the computing device 102, is configured to improve grammar, structure, and style of the SOAP notes and clinical progress notes without altering the content of the information received from the counselor.

At step 312, the treatment plan generation module 214, at the computing device 102, is configured to generate a treatment plan based on information received in response to the sets of questions and any existing treatment plan or associated score data. The treatment plan generation module 214 further utilizes clinical progress notes and score data to provide one or more treatment plan modifications to the counselor for incorporation into the treatment plan. The treatment plan is for a predefined duration based on the information of the client including score data and clinical progress notes.

At step 314, the coaching document generation module 216, at the computing device 102, is configured to generate documentation feedback and improvement suggestions related to clinical note quality, formatting, and compliance, and enable the supervisor to guide the counselor.

At step 316, the feedback module 218, at the computing device 102, is configured to analyze the clinical progress notes and provide feedback to the counselor for improving the quality, structure, and compliance of the notes.

Further, the query module 220, at the computing device 102, is configured to provide a questionnaire comprising a plurality of categories. Each category comprising a set of questions to assess progress of the client. The query module 220 is further configured to enable the clinician to input a score for each question of each category based on analyzing the response of the client to each question. Further, the scoring module 222 is configured to apply predefined scoring guidelines for the input score to generate the score data.

At step 318, the personal recovery plan module 224, at the computing device 102, is configured to generate a client-specific strategic recovery document. The recovery document is a persistent, client-owned record transferable across counseling sessions and service providers, and further configured to enable client participation in treatment planning using the recovery document.

At step 320, the assessment analysis module 226 is configured to analyze score data associated with the client and generate one or more recommendations for influencing a plurality of variables of the questionnaire. At step 322, the personal recovery assistant module 228 is configured to monitor the score data associated with the client to detect indicators of non-compliance with recovery protocols and to provide the client with personalized feedback and suggestions for improving adherence to the treatment plan.

Advantageously, the system 100 provides both quantitative data and qualitative data that help in understanding the client's progress and treatment requirements. The quantitative data is the score data assigned to the client data and the qualitative data is the notes provided by the clinician. Further, the qualitative and quantitative data approach enhances predictive capabilities and supports individualized treatment plans. The system 100 ensures accessibility across various user environments by maintaining compatibility with existing technology infrastructures in clinics and other healthcare settings. The system 100 integrated with the existing electronic health records (EHR) system ensures the real-time synchronization of the client data across multiple platforms. Further, the integration supports streamlined workflows and ensures the data used for assessments is always accurate.

Further, the system 100 facilitates real-time alerts for missed appointments, irregularities in behavior, or medication adherence. The system 100 is compatible with both the iOS and the android devices which enables the user to access the system 100 across different devices. The system 100 utilizes the AWS and Microsoft Azure platforms to ensure compliance with healthcare regulations (HIPAA) and enable large-scale deployment across treatment facilities.

The system 100 automates key documentation tasks and provides AI-driven suggestions for SOAP notes and treatment plans. The automation function reduces the administrative burden on clinicians and improves workflow efficiency. Additionally, the system 100 incorporates the data and guidelines from federal and state regulatory bodies, for example, the Drug Enforcement Administration (DEA), the Licensing and Regulatory Affairs (LARA) department, and the Substance Abuse and Mental Health Services Administration (SAMHSA), into the AI logic. The integration ensures that the recommendations generated are not only evidence-based but also fully compliant with regulatory requirements. The system 100 is designed with a modular architecture. The modular architecture enables new features or compliance updates to be added without overhauling the entire system. The modulator approach also supports incremental innovation, enabling independent enhancements or replacements of specific components based on technological advancements or regulatory changes. The system 100 addresses both the therapeutic and diagnostic domains by demonstrating the practical utility and clinical relevance. Further, the system 100 emphasizes clinical and functional advancements, specifically in improving treatment outcomes by integrating automation and personalization in treatment and diagnostic processes.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device, or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A system for managing treatment plans and clinical documentation, comprising:
one or more user devices including at least one first user device associated with a client, at least one second user device associated with a clinician, at least one third user device associated with a representative of an organization and at least one fourth user device associated with a supervisor;
at least one database comprises a client data, a clinician data, a score data of each client assessment, and data related to clinical documentation including SOAP notes and clinical progress notes, and
at least one computing device in communication with at least one database and the user devices via a network, wherein the computing device comprises one or more artificial intelligence (AI) modules, wherein the computing device comprises at least one memory configured to store a set of program modules and at least one processor configured to execute one or more program modules, wherein the modules comprise:
a data collection module configured to present a predefined first set of questions at the second user device and collect information for generating SOAP notes, wherein the data collection module is further configured to present a predefined second set of questions and collect information for generating clinical progress notes,
a secondary data collection module configured to present a predefined third set of questions, wherein the third set of questions are configured to collect information to elicit clarification from the counselor without requiring a response from the client,
a review module configured to present generated SOAP notes and clinical progress notes and configured to enable the counselor to customize the notes through one or more options including review, and edit, and enable the counselor to approve, and submit the SOAP notes and clinical progress notes,
a documentation module configured to improve grammar, structure, and style without altering a content of the information received from the counselor, and
a treatment plan generation module configured to generate a treatment plan based on information received in response to the set of questions and any existing treatment plan or associated score data.

2. The system of claim 1, wherein the modules further comprise:
a coaching document generation module configured, upon request, to provide a supervisor with documentation feedback and improvement suggestions related to clinical note quality, formatting, and compliance, the feedback is being generated to enable the supervisor to guide the counselor, and
a feedback module configured to analyze clinical progress notes and provide feedback to the counselor for improving the quality, structure, and compliance of the clinical progress notes.

3. The system of claim 1, wherein the modules further comprise:
a query module configured to provide a questionnaire comprising a plurality of categories, wherein each category comprises a set of questions to assess progress of the client, and wherein the query module is configured to enable the clinician to input a score for each question of each category based on analyzing response of client to each question, and
a scoring module configured to apply predefined scoring guidelines for the input score and generate the score data.

4. The system of claim 1, wherein the modules further comprise:
a personal recovery plan module configured to generate a client-specific strategic recovery document, wherein the recovery document is a persistent, client-owned record transferable across counselling sessions and service providers, and wherein the personal recovery plan module is further configured to enable client participation in treatment planning using the recovery document,
an assessment analysis module configured to analyze score data associated with the client and generate one or more recommendations for influencing a plurality of variables of the questionnaire, and
a personal recovery assistant module configured to monitor score data associated with the client to detect indicators of non-compliance with recovery protocols, and to provide the client with personalized feedback and suggestions for improving adherence to the treatment plan.

5. The system of claim 1, wherein the data collection module is configured to generate the clinical progress note and SOAP note using a predefined style guide, grammar enhancement tools, and one or more evidence-based documentation inputs.

6. The system of claim 1, wherein the treatment plan generation module is further configured to utilize clinical progress notes and score data to provide one or more treatment plan modifications to the counselor for incorporation into the treatment plan.

7. The system of claim 1, wherein the treatment plan is for a predefined duration based on the information of the client including score data and clinical progress notes.

8. A method for managing treatment plans and clinical documentation, comprising:
providing one or more user devices, a database, and at least one computing device in communication with the database and user devices via a network, wherein the user devices include at least one first user device associated with a client, at least one second user device associated with a clinician, at least one third user device associated with a representative of an organization, and at least one fourth user device associated with a supervisor;
storing, in the database, client data, clinician data, score data of each client assessment, and data related to clinical documentation including SOAP notes and clinical progress notes;
presenting, at the computing device via a data collection module, a predefined first set of questions at the second user device and collecting information for generating SOAP notes;
presenting, at the computing device via the data collection module, a predefined second set of questions and collecting information for generating clinical progress notes;
presenting, at the computing device via a secondary data collection module, a predefined third set of questions configured to elicit clarification from the counselor without requiring a response from the client;
presenting, at the computing device via a review module, generated SOAP notes and clinical progress notes to the counselor and enabling customization through one or more options including review and edit, and enabling the counselor to approve and submit the SOAP notes and clinical progress notes;
improving, at the computing device via a documentation module, grammar, structure, and style without altering a content of the information received from the counselor, and
generating, at the computing device via a treatment plan generation module, a treatment plan based on information received in response to the sets of questions and any existing treatment plan or associated score data.

9. The method of claim 8, further comprising step of: generating, at the computing device via a coaching document generation module, documentation feedback and improvement suggestions related to clinical note quality, formatting, and compliance, and enabling the supervisor to guide the counselor.

10. The method of claim 8, further comprising step of analysing, at the computing device via a feedback module, the clinical progress notes and providing feedback to the counselor for improving the quality, structure, and compliance of the clinical progress notes.

11. The method of claim 8, further comprising steps of:
providing, at the computing device via a query module, a questionnaire comprising a plurality of categories, each comprising a set of questions to assess progress of the client, and enabling the clinician to input a score for each question of each category based on analyzing response of client to each question, and
applying, at the computing device via a scoring module, predefined scoring guidelines for the input score to generate the score data.

12. The method of claim 8, further comprising steps of:
generating, at the computing device via a personal recovery plan module, a client-specific strategic recovery document, wherein the recovery document is a persistent, client-owned record transferable across counselling sessions and service providers, and wherein the personal recovery plan module is further configured to enable client participation in treatment planning using the recovery document;
analysing, at the computing device via an assessment analysis module, score data associated with the client and generating one or more recommendations for influencing a plurality of variables of the questionnaire, and
monitoring, at the computing device via a personal recovery assistant module, the score data associated with the client to detect indicators of non-compliance with recovery protocols and providing the client with personalized feedback and suggestions for improving adherence to the treatment plan.

13. The method of claim 8, wherein the data collection module generates the clinical progress note and SOAP note using a predefined style guide, grammar enhancement tools, and one or more evidence-based documentation inputs.

14. The method of claim 8, wherein the treatment plan generation module utilizes clinical progress notes and score data to provide one or more treatment plan modifications to the counselor for incorporation into the treatment plan.

15. The method of claim 8, wherein the treatment plan is for a predefined duration based on the information of the client, including score data and clinical progress notes.
